(19) United States
(12) Patent Application Publication
(10) Pub. No.: US 2024/0180910 A1
(43) Pub. Date: Jun. 6, 2024

(54) METHOD FOR TREATING CANCER WITH A CHEMOTHERAPEUTIC MODIFIER

(71) Applicant: Elion Oncology, Inc., Bethesda, MD (US)

(72) Inventors: Lorenzo Leoni, Lodrino (CH); Khalid Islam, Lugano (CH)

(21) Appl. No.: 18/529,271

(22) Filed: Dec. 5, 2023

Related U.S. Application Data
(60) Provisional application No. 63/386,271, filed on Dec. 6, 2022.
(60) Provisional application No. 63/513,525, filed on Jul. 13, 2023.

(51) Int. Cl. A61K 31/513; A61K 9/00; A61K 31/7068; A61P 35/00

(52) U.S. Cl. CPC A61K 31/513; A61K 9/0053; A61K 31/7068; A61P 35/00

(57) ABSTRACT

The present invention relates to a method for treating cancer in a human subject. The method comprises: (a) orally administering eniluracil at a dose 30-60 mg/day to the subject; (b) at least after 10 hours of step (a), orally administering capecitabine at a dose about 140-700 mg/day or at a dose providing a 5-FU exposure (AUC) of approximately 1500 h*ng/ml or higher to the subject for 2-14 consecutive days; and (c) 3-14 days after step (b), repeat the steps (a) and (b). The present invention is suitable to treat colorectal cancer, gastrointestinal tract tumor, breast cancer, pancreatic cancer, head and neck cancer, lung cancer, or advanced biliary tract cancer.

### CROSS REFERENCE TO RELATED APPLICATIONS

This application claims the benefit of priority from U.S. Provisional Patent Application Nos. 63/386,271, filed on Dec. 6, 2022, and 63/513,525, filed on Jul. 13, 2023, which are both hereby incorporated by reference in their entirety.

### FIELD OF THE INVENTION

The present invention relates to a method of treating cancer, by orally administering eniluracil and capecitabine to a cancer patient. The method provides optimal dosing schedules and amounts of eniluracil and capecitabine to improve the efficacy-safety profile of capecitabine.

### BACKGROUND

5-Fluorouracil (5-FU) is one of the most widely used chemotherapeutic drugs in the treatment of a variety of tumors. 5-FU must be activated by metabolic conversion to fraudulent uridine nucleotides and deoxyuridine nucleotides that interfere with DNA synthesis and RNA functions. Because 5-FU differs from uracil, its natural counterpart, by only a fluorine substitution in the 5-position, it is readily activated in cancer patients. Unfortunately, its structural similarity to uracil also accounts for its rapid and extensive conversion to breakdown products that have no antitumor activity. The first step in the breakdown of 5-FU is through the drug metabolizing enzyme, dihydropyrimidine dehydrogenase (DPD: EC 1312, uracil reductase).

DPD is a ubiquitous enzyme that is the first and the rate-limiting step in the degradation (inactivation) of 5-FU. DPD is abundantly expressed in the gastrointestinal tract and the liver but also exists in other cells within the body and in cancer cells. The oral bioavailability of 5-FU is poor because of the extensive and rapid first-pass metabolism that occurs from the abundant amounts of DPD in the liver.

5-Ethynyluracil, also referred to as eniluracil, is a DPD inhibitor that is an irreversible inactivator of DPD thus reducing or eliminating the metabolic inactivation of 5-FU. Due to the structural similarity between eniluracil and 5-FU, eniluracil is a substrate for DPD. The co-administering of eniluracil with 5-FU restores the oral bioavailability of 5-FU. Eniluracil has been shown to be safe when doses up to 50 g per day were administered for seven days. [1]

Capecitabine, the first oral fluoropyrimidine, was introduced as a promising alternative therapy that does not require infusion and yet provides the benefits of 5-FU therapy. Capecitabine is a prodrug, which is converted to 5-FU via three enzymatic steps. Thymidylate phosphorylase (TP) plays a key role in the conversion of capecitabine to its active metabolite and may be present in higher concentrations in some malignant tissues [2]. Clinical trials have shown an improved side effect profile when comparing capecitabine therapy to 5FU/LV therapy and includes decreased stomatitis, diarrhea, nausea and neutropenic sepsis. The FDA approved capecitabine (Xeloda®) for the treatment of Dukes' stage C colon cancer, metastatic colorectal cancer and metastatic breast cancer. However, use of capecitabine at the approved doses has shown substantially higher rates of hyperbilirubinemia and hand-foot syndrome [3]. After capecitabine oral dosing at 1255 mg/m², the PK parameters for capecitabine, 5-FU, 5-DFCR and 5′-DFUR indicate a rapid elimination (as shown below in Table 1). The observed exposure (AUC) of 5-FU is approximately 1630 h*ng/mL. FBAL has slightly extended PK profile with a half-life of 3.23 hours and an AUC of 30-35,000 h*ng/ml [4].

TABLE 1

| Parameter | Capecitabine | 5′-DFCR | 5′DFUR | 5-FU | FUH₂ | FBAL |
|---|---|---|---|---|---|---|
| Cₘₐₓ | 3.99 | 1.71 | 9.37 | 0.709 | 0.442 | 5.32 |
| (μg/mL) | (56%) | (236%) | (94%) | (87%) | (103%) | (26%) |
| (h) | 1.50 | 2.00 | 2.00 | 2.00 | 2.28 | 3.34 |
| tₘₐₓ | (0.78-2.17) | (0.78-4.08) | (1.28-4.08) | (1.28-4.08) | (2.00-4.08) | (3.00-5.58) |
| AUC₀₋ₜ | 7.29 | 3.97 | 19.9 | 1.62 | 1.20 | 30.0 |
| (μg · h/mL) | (32%) | (175%) | (57%) | (62%) | (153%) | (20%) |
| AUC_{0-∞} | 7.40 | 5.21 | 21.7 | 1.63 | 2.15 | 35.2 |
| (μg · h/mL) | (34%) | (140%) | (63%) | (74%) | (67%) | (27%) |
| t_{1/2} | 0.85 | 1.31 | 0.66 | 0.76 | 1.14 | 3.23 |
| (h) | (88%) | (80%) | (17%) | (25%) | (26%) | (40%) |

Geometric means (CV) are reported for Cₘₐₓ, AUC₀₋ₜ, and AUC_{0-∞}, Median values (min-max) are reported for tₘₐₓ, Arithmetic means (CV) are reported for t_{1/2}.

Descriptive Statistics on the Pharmacokinetic Parameters Estimated on Day 14 after Administration of Capecitabine (1255 mg/m²) in 8 Cancer Patients [4].

### BRIEF SUMMARY

In one aspect, the present disclosure provides a method for treating cancer in a human subject, the method comprising: (a) orally administering eniluracil at a dose of 30-60 mg/day to the subject; (b) at least after 10 hours of step (a), orally administering a fixed dose of capecitabine at about 140-700 mg/day to the subject for 2 to 14 consecutive days or a dose of capecitabine providing a 5-FU exposure (AUC) of approximately 1500 h*ng/ml or higher; and (c) 3 to 14 days after step (b), repeat the steps (a) and (b).

In a further embodiment and in accordance with the above, eniluracil is administered at about 40 mg/day.

In a further embodiment and in accordance with any of the above, capecitabine is administered at about 160-500 mg/day.

In a further embodiment and in accordance with any of the above, step (b) occurs at least after 17 hours of step (a). In some other further embodiments, step (b) occurs 10-24 hours after step (a).

In a further embodiment and in accordance with any of the above, step (c) occurs 7 days after step (b).

In a further embodiment and in accordance with any of the above, the cancer is colorectal cancer, gastrointestinal tract tumor, breast cancer, pancreatic cancer, head and neck cancer, lung cancer, or advanced biliary tract cancer.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.”, “FIG.”, “Figure”, “Figures”, “Figs.”, and “FIGs.” herein) of which:

FIG. 1 depicts the antitumor efficacy of capecitabine and eniluracil in mono- and combination therapy. Median relative tumor volumes over time are shown for Groups 1-6 of the in vivo efficacy experiment.

FIG. 2 depicts mean AUC_{0-∞} levels of 5-FU observed at day 2 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 3 depicts Cₘₐₓ data for 5-FU observed at day 2 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 4 depicts mean AUC_{0-∞} levels of 5-FU observed at day 8 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 5 depicts Cₘₐₓ data for 5-FU observed at day 8 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 6 depicts mean AUC_{0-∞} levels of FBAL observed at day 2 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 7 depicts Cₘₐₓ data for FBAL observed at day 2 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 8 depicts mean AUC_{0-∞} levels of FBAL observed at day 8 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 9 depicts Cₘₐₓ data for FBAL observed at day 8 following treatment with eniluracil and capecitabine. Solid line indicates corresponding PK parameter data from the Xeloda FDA regulatory submission. Dashed line indicates corresponding PK parameter data from the Xeloda EMA regulatory submission.

FIG. 10 depicts mean AUC_{0-∞} levels of capecitabine observed at day 2 following treatment with eniluracil and capecitabine. Solid line indicates corresponding PK parameter data from the Xeloda FDA regulatory submission.

FIG. 11 depicts Cₘₐₓ data for capecitabine observed at day 2 following treatment with eniluracil and capecitabine. Solid line indicates corresponding PK parameter data from the Xeloda FDA regulatory submission.

FIG. 12 depicts mean AUC_{0-∞} levels of DFCR observed at day 2 following treatment with eniluracil and capecitabine. Solid line indicates corresponding PK parameter data from the Xeloda FDA regulatory submission.

FIG. 13 depicts Cₘₐₓ data for DFCR observed at day 2 following treatment with eniluracil and capecitabine. Solid line indicates corresponding PK parameter data from the Xeloda FDA regulatory submission.

FIG. 14 depicts mean AUC_{0-∞} levels of DFUR observed at day 2 following treatment with eniluracil and capecitabine. Solid line indicates corresponding PK parameter data from the Xeloda FDA regulatory submission.

FIG. 15 depicts Cₘₐₓ data for DFUR observed at day 2 following treatment with eniluracil and capecitabine. Solid line indicates corresponding PK parameter data from the Xeloda FDA regulatory submission.

### BRIEF DESCRIPTION OF THE TABLES

Table 1 depicts descriptive statistics on the pharmacokinetic parameters estimated on Day 14 after administration of capecitabine (1255 mg/m²) in 8 cancer patients.

Table 2 depicts the design of an in vivo efficacy experiment with patient-derived colon cancer xenograft CXF 280 implanted subcutaneously in athymic nude mice.

Table 3 depicts the anti-tumor efficacy of capecitabine (alone, or in combination with eniluracil) expressed as minimum T/C value in percent. The minimum T/C value recorded for a test group represents the maximum antitumor efficacy for the respective treatment.

Table 4 depicts mean Tₘₐₓ and T_{1/2} data for 5-FU at day 2 following treatment with capecitabine (alone, or in combination with eniluracil) for the various cohorts.

Table 5 depicts mean Cₘₐₓ data for 5-FU at day 2 following treatment with capecitabine (alone, or in combination with eniluracil) for the various cohorts.

Table 6 depicts mean AUCₗₐₛₜ data for 5-FU at day 2 following treatment with capecitabine (alone, or in combination with eniluracil) for the various cohorts.

Table 7 depicts a summary of the patient cohorts (1, 2A, 3, and 4), including: gender, age, cancer type, dose of PCS6422, dose of capecitabine, and number of cycles completed.

Table 8 depicts a summary of ADRs reported in ≥5% of patients with colon cancer tested with Xeloda monotherapy or i.v. 5-FU/LV in the adjuvant setting.

Table 9 depicts a summary of adverse events (Grade 2 or higher) observed in cohorts 1, 2A, 3, and 4.

Table 10 depicts data regarding the RECIST analysis of the observed lesion(s) for patients enrolled in the clinical trial.

### DETAILED DESCRIPTION OF THE INVENTION

The inventors have compared the anti-tumor efficacy of the combination of eniluracil and capecitabine using a 7/7 (capecitabine 7 days on and 7 days off) dosing schedule with capecitabine alone using the conventional 14/7 (capecitabine 14 days on and 7 days off) schedule in a preclinical xenograft model. The inventors discovered that pretreatment with eniluracil allows for the complete elimination of eniluracil in the mice prior to treatment with capecitabine and enhances the anti-tumor activity of capecitabine without a gross impact on its safety and tolerability profile. Pretreatment with 10 mg/kg oral eniluracil prior to administration of 30 mg/kg or 15 mg/kg of oral capecitabine resulted in comparable efficacy to that of 300 mg/kg of oral capecitabine alone in mice. Notably, the total cumulative dose of capecitabine in the combination groups was 13.3 and 26.6-fold lower compared to the capecitabine alone group.

Based on inventors' xenograft model efficacy results, in conjunction with the knowledge of previous preclinical and clinical antitumor efficacy using eniluracil and 5-FU combination therapy, the inventors discovered optimal dosing schedules and amounts of eniluracil and capecitabine for treating cancers in patients where fluoropyrimidine therapy alone is preferred.

The present application provides a method for treating cancer in a patient, wherein the cancer is a cancer type in which 5-FU or 5-FU prodrugs such as capecitabine have activity. The method comprises the steps of: (a) first orally administering eniluracil at a dose about 30-60 mg/day to the patient, (b) at least after 10 hours of step (a), orally administering capecitabine at a dose about 140-700 mg/day to the patient for 6-14 consecutive days or orally administering capecitabine at a dose providing a of 5-FU exposure (AUC) of approximately 1500 h*ng/ml or higher; and (c) 7-14 days after step (b), repeat the steps (a) and (b).

In step (a) of the method, at least about 30 mg/day or at least about 40 mg/day of eniluracil is administered to a patient. For example, about 40 mg/day, or about 50 mg/day, or about 60 mg/day, of eniluracil is administered to a patient. Eniluracil is first administered (i.e., pre-dosed prior to capecitabine) to a patient to substantially eliminate the DPD activity in both nervous and non-nervous tissues in the patient, before administration of the 5-FU prodrug capecitabine. By “substantially eliminate”, it means that the level of DPD activity in both nervous and non-nervous tissues in the patient is reduced to less than 10%, and preferably to less than 5%, or less than 1% of the baseline DPD activity in the patient prior to administration of eniluracil.

In step (b) of the method, capecitabine is administered between 10-24 hours after administering eniluracil. For example, at least after 10 hours, after 12 hours, after 16 hours, after 17 hours, and no longer than 24 hours after administering eniluracil, capecitabine is administered. The dosing schedule and the capecitabine amount are selected to allow eniluracil to be substantially cleared from the patient prior to capecitabine administration, so that capecitabine is present in molar excess (e.g., at least 5 fold, at least 10 fold, at least 25 fold, at least 50 fold, or at least 100 fold) relative to the level of eniluracil remaining in the patient at the time capecitabine is administered. Accordingly, the potential interference by eniluracil with the metabolic activation of capecitabine is minimized, and the anti-tumor efficacy of capecitabine is thereby not interfered by eniluracil.

Capecitabine at a dose between approximately 140 to 700 mg/day, is administered to the patient. For example, capecitabine is administered at a dose of 140-700 mg/day, 160-700 mg/day, 140 to 500 mg/day, 150-500 mg/day, 200-500 mg/day, 140-450 mg/day, 150-450 mg/day, 160-450 mg/day, 140-400 mg/day, 150-400 mg/day, or 300-400 mg/day.

Capecitabine could also be administered at a dose providing 5-FU exposure (AUC) of approximately 1500 h*ng/ml or higher.

Lower amount of capecitabine is non-efficacious or only partially efficacious. Higher amount of capecitabine is expected to cause toxicity.

Capecitabine can be administered once a day, or more than once a day to the patient. Preferably, capecitabine is administered twice a day. Capecitabine is administered to the patient for 2-14 consecutive days, for example, for 2, 3, 4, 5, 6, 7, 8, 9, 10, 11, 12, 13 or 14 consecutive days.

In step (c) of the method, after the patient is rested for at least 3 to 14 days, for example, or after 3, 4, 5, 6, or 7 days, or after any of 7-14 days, the patient receives the next cycle of eniluracil and capecitabine which follows the steps (a) and (b).

The steps (a)-(c) are repeated based on the discretion of the physician evaluation of response and side effects. A physician may often stop treating a patient if the patient either has a complete response or when the disease progresses in the patient. A complete response means that the tumor is no longer present. The progress of the disease means that the tumor starts to enlarge or the tumor is no longer stable. A tumor is stable means that the tumor is not progressing and not regressing.

The present invention is useful in treating all cancers that capecitabine is used, including but not limited to colorectal cancer, gastrointestinal tract tumor, breast cancer, pancreatic cancer, head and neck cancer, lung cancer, and advanced biliary (bile) tract cancer.

The present invention is useful in treating a mammal subject, such as humans, horses, and dogs. The present invention is particularly useful in treating humans.

The present method uses an optimal dosing schedule and optimal amounts of both eniluracil and capecitabine, which provide an efficacious treatment and is safe. The total cumulative dose of capecitabine administered in the present method is lower than the effective dose of capecitabine if used alone without eniluracil. This is beneficial because high doses of capecitabine treatment may cause side effects resulting in the need to decrease the dose or discontinue treatment as well as resistance to therapy as evidenced by shorter progress-free survival and lower response rate in patients [5]. The present method is also useful in treating patients with tumor resistance to either 5-FU or capecitabine therapy.

### Examples

The following examples further illustrate the present invention. These examples are intended merely to be illustrative of the present invention and are not to be construed as being limiting.

### Example 1. Antitumor Efficacy of Eniluracil and Capecitabine in Mice

### A. Objectives

This study was to assess the antitumor efficacy of eniluracil in combination with different doses of capecitabine in the patient-derived colon xenograft CXF 280 subcutaneously implanted in immunodeficient athymic nude mice.

### B. Mice Model

Human tumor explants directly transplanted from patients to nude mice, and passaged subcutaneously termed patient-derived tumor xenografts (PDXs), retain most of the characteristics of the parental patient tumors including histology and sensitivity to anti-cancer drugs. Studies have shown that PDXs passaged in nude mice correctly replicate the response of the donor patient to standard cytotoxic anti-cancer drugs in >90% of cases. [6], [7], [8].

### C. Study Design

The study consisted of an in vivo efficacy experiment with the patient-derived colon cancer xenograft CXF 280 implanted subcutaneously in athymic nude mice.

This experiment consisted of six groups with six animals each. Animals were dosed in monotherapy with capecitabine at 300 or at 30 mg/kg/day daily for 2 weeks with a one-week dosing break followed by another 2 weeks of daily dosing (14 days ON and 7 days OFF). In combination therapies, capecitabine was dosed at 30, 15 or 7.5 mg/kg/day on days 1-7, 15-21 and 29-35 (7 days ON and 7 days OFF) as well as with eniluracil at 10 mg/kg/day on days 1, 3, 5, 15, 17, 19, 29, 31, and 33.

Combination therapies with capecitabine and eniluracil scheduled for the same dosing day were administered with a six-hour dosing delay after dosing with eniluracil in order to ensure that all the eniluracil is eliminated in the mouse prior to capecitabine administration.

All therapies were administered via oral gavage (p.o.) over the course of 35 days followed by an observation period of one week until day 42 of the experiment.

Table 2 shows the design of the experiment.

TABLE 2

| | | Total Daily | | | |
|---|---|---|---|---|---|
| Group | | Dose | Schedule | Therapy | No. of |
| ID | Therapy | [mg/kg/day] | [Dosing days] | Route | Animals |
| 1 | Control | 10 ml/kg | 1, 3, 5, 15, 17, 19, 29, 31, 33 (h: 0) | p.o. | 6 |
| | Vehicle 1 | // | // | // | |
| | // | 10 ml/kg | 1-7, 15-21, 29-35 (h: 6) | p.o. | |
| | Control | | | | |
| | Vehicle 2 | | | | |
| 2 | Capecitabine | 300 | 1-14, 22-35 | p.o. | 6 |
| 3 | Capecitabine | 30 | 1-14, 22-35 | p.o. | 6 |
| 4 | Capecitabine | 30 | 1-7, 15-21, 29-35 (h: 6) | p.o. | 6 |
| | // | // | // | // | |
| | Eniluracil | 10 | 1, 3, 5, 15, 17, 19, 29, 31, 33 (h: 0) | p.o. | |
| 5 | Capecitabine | 15 | 1-7, 15-21, 29-35 (h: 6) | p.o. | 6 |
| | // | // | // | // | |
| | Eniluracil | 10 | 1, 3, 5, 15, 17, 19, 29, 31, 33 (h: 0) | p.o. | |
| 6 | Capecitabine | 7.5 | 1-7, 15-21, 29-35 (h: 6) | p.o. | 6 |
| | // | // | // | // | |
| | Eniluracil | 10 | 1, 3, 5, 15, 17, 19, 29, 31, 33 (h: 0) | p.o. | |

Control vehicle 1 capecitabine: water for injection; control vehicle 2 and eniluracil: PBS pH 8.5

### D. Results

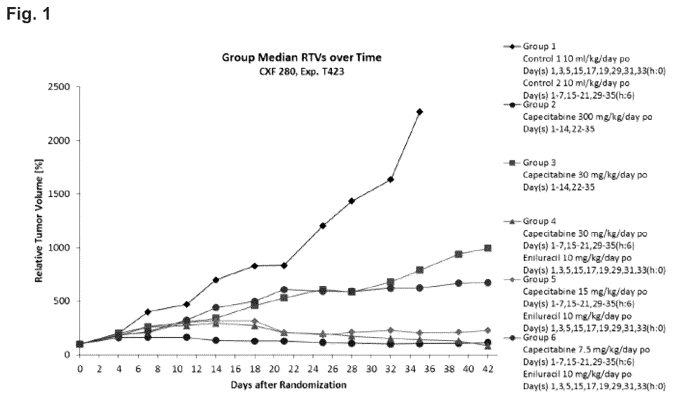

FIG. 1 shows the antitumor efficacy of capecitabine and eniluracil in mono- and combination therapy. Median relative tumor volumes over time are shown in Groups 1-6. Relative tumor volumes (RTVs) for Day x were calculated by dividing the absolute individual tumor volume on Day x (Tx) by the absolute individual tumor volume of the same tumor on the day of randomization (Tr) multiplied by 100%.

Tumor growth inhibition was determined by comparison of the median RTV of the test groups (T) with the vehicle control group (C) and was expressed as minimum T/C value in percent. The minimum T/C value recorded for a test group during an experiment represents the maximum antitumor efficacy for the respective treatment.

The anti-tumor efficacy is also shown in Table 3 by calculating minimum T/C value (%) at Day 35.

TABLE 3

| | | Total Daily | | Minimum | |
|---|---|---|---|---|---|
| Group | | Dose | Schedule | T/C | Efficacy |
| ID | Therapy | [mg/kg/day] | [Dosing days] | (%) | Rating |
| 1 | Control | 10 ml/kg | 1, 3, 5, 15, 17, 19, 29, 31, 33 (h: 0) | n/a | n/a |
| | Vehicle 1 | // | // | | |
| | // | 10 ml/kg | 1-7, 15-21, 29-35 (h: 6) | | |
| | Control | | | | |
| | Vehicle 2 | | | | |
| 2 | Capecitabine | 300 | 1-14, 22-35 | 4.5 | ++++ |
| 3 | Capecitabine | 30 | 1-14, 22-35 | 34.9 | + |
| 4 | Capecitabine | 30 | 1-7, 15-21, 29-35 (h: 6) | 6.2 | +++ |
| | // | // | // | | |
| | Eniluracil | 10 | 1, 3, 5, 15, 17, 19, 29, 31, 33 (h: 0) | | |
| 5 | Capecitabine | 15 | 1-7, 15-21, 29-35 (h: 6) | 9.0 | +++ |
| | // | // | // | | |
| | Eniluracil | 10 | 1, 3, 5, 15, 17, 19, 29, 31, 33 (h: 0) | | |
| 6 | Capecitabine | 7.5 | 1-7, 15-21, 29-35 (h: 6) | 27.5 | + |
| | // | // | // | | |
| | Eniluracil | 10 | 1, 3, 5, 15, 17, 19, 29, 31, 33 (h: 0) | | |

The above results show that monotherapy with capecitabine at 300 mg/kg/day in mice achieved high antitumor activity and was significantly more efficacious than monotherapy with 30 mg/kg/day. Combination therapies of capecitabine with eniluracil were most efficacious with 30 or 15 mg/kg/day of capecitabine, reaching very high levels of tumor growth inhibition at a much lower cumulative dose due to the addition of eniluracil and a higher level of capecitabine exposure than the 7.5 mg/kg/day group. All combination therapies of capecitabine (30, 15 and 7.5 mg/kg/day) with eniluracil were more efficacious than the monotherapy of capecitabine with 30 mg/kg/day, at a much lower cumulative dose due to the modified dosing schedule.

### Example 2. Clinical Study Protocol

### A. Objectives:

To evaluate the safety, dose-limiting toxicities (DLTs), and maximum tolerated dose (MTD) of capecitabine administered to patients with advanced, refractory gastrointestinal tract (GI) tumors using a 7 days on +7 days off capecitabine regimen ˜24 hours after a single, fixed, oral 40 mg dose of eniluracil.

### B. Study Design:

This study is an open label, multi-center study in patients who have advanced, relapsed refractory GI cancer or are not relapsed/refractory but are intolerant to other therapies who, in the judgment of investigators, are candidates for fluoropyrimidine monotherapy.

### C. Inclusion Criteria:

Patients meeting the following criteria will be eligible for the study:
1. Has advanced, metastatic or unresectable GI tract tumors that are refractory or intolerant to existing available therapies and for whom the investigator recommends fluoropyrimidine monotherapy.
2. Has measurable disease in accordance with Respond Evaluation Criteria in Solid Tumors (RECIST) guidelines (Version 1.1).
3. Is aged ≥18 years.
4. Has not received treatment with intravenous (IV) 5 FU or oral 5 FU analogs in the 4 weeks preceding enrollment.
5. Has Eastern Cooperative Oncology Group (ECOG) Performance Status 0 2 at study entry.
6. Has adequate bone marrow, liver, and renal function as assessed by the following laboratory requirements conducted within 7 days before starting study treatment:
   a. peripheral ANC of ≥1.5×109/L;
   b. platelet count of ≥75×109/L without growth factor/transfusion;
   c. hemoglobin ≥8.5 g/dL without growth factor/transfusion;
   d. estimated glomerular filtration rate >50 mL/min;
   e. total bilirubin <2× upper limit of normal (ULN); <5×ULN if patient has liver metastases, biliary tract cancer; or ≤3×ULN if the patient has Gilbert's disease;
   f. Aspartate aminotransferase (AST) or alanine aminotransferase (ALT)<2.5× ULN, with liver metastasis <5×ULN; and
   g. international normalized ratio (INR)<1.5.
7. Has a life expectancy of at least 12 weeks.
8. Female patients of childbearing potential and male patients with partners capable of reproduction must agree to use an effective contraceptive method as described below from the time of Screening through 60 days after the last dose of capecitabine:
   a highly effective method of contraception, including hormonal contraceptives (e.g., combined oral contraceptives, patch, vaginal ring, injectables, and implants), intrauterine device, or intrauterine system, vasectomy, or tubal ligation; OR
   an effective double-barrier contraceptive method (2 of the following: male condom, female condom, cervical cap, diaphragm, or contraceptive sponge); OR
   abstain from sex during study participation and for 60 days after the last dose of capecitabine.
9. Females of childbearing potential must have a negative serum pregnancy β human chorionic gonadotropin pregnancy test result within 3 days before the first study treatment administration. Female patients who are surgically sterilized with a hysterectomy and/or bilateral oophorectomy or who are postmenopausal (≥12 months of amenorrhea and at least 55 years of age) may have the β human chorionic gonadotropin pregnancy test waived.
10. Willingly provides written, informed consent.
11. Has resolution or stabilization of acute toxicity from prior therapy to Grade <2—except Grade 2 neuropathy.
12. If patient has human immunodeficiency virus (HIV) infection, it is controlled with undetectable viral load with antiretroviral treatment.
13. If patient has hepatitis C infection and received antiviral treatment, has a negative viral load at Screening.
14. If patient has chronic hepatitis B infection and is receiving antiviral treatment, has a negative viral load at Screening.
15. Is willing and able to comply with all protocol required visits and assessments.

### D. Exclusion Criteria:

Patients meeting any of the following criteria will not be allowed to enroll in this study:
1. Is unable to take oral medication or malabsorption syndromes potentially interfering with medication absorption (e.g., short bowel syndrome or chronic, partial bowel obstruction).
2. Has history or presence of clinically significant abnormal 12 lead ECG results, in the investigator's opinion.
3. Has current brain metastasis.
4. Has prolonged QTc (with Fridericia's correction) of >480 msec in men and women performed at Screening.
5. Has a history of prolonged QTc interval, ventricular tachycardia/fibrillation or significant ventricular arrhythmia, or Torsades de Pointes, or a history of ventricular ablation for arrhythmia.
6. Has congenital long QT syndrome or a family history of long QT syndrome.
7. Has other clinically significant cardiac disease including, but not limited to, uncontrolled angina, myocardial ischemia or infarction within 6 months, congestive heart failure >Class II per the New York Heart Association, or history of myocarditis.
8. Has an electrolyte disturbance, such as uncorrected hypokalemia/hyperkalemia, hypomagnesemia, or hypocalcemia. Patients can be enrolled following successful correction of an electrolyte disturbance.
9. Is currently using any drugs included in the prohibited medications list in the protocol (including those that can prolong QTc) that cannot be discontinued.
10. Has known hypersensitivity to any of the components of study treatments.
11. Has other primary cancer requiring treatment within the last 3 years, except for cervical intraepithelial neoplasia, ductal carcinoma in situ, or completely excised squamous or basal cell carcinoma.
12. Is a pregnant or lactating female.
13. Had major surgery, open biopsy, or significant traumatic injury within 4 weeks prior to the first dose of study treatment.
14. Is receiving or has received any investigational treatment within 4 weeks prior to study entry, or participating in another clinical study.
15. Has known DPD deficiency.

### E. Drug Administration and Duration:

Eniluracil (40-50 mg) is orally administrated at Day 1. After 12-24 hours, capecitabine is orally administered. Capecitabine is given twice a day at 140-600 mg/day for a total of 7 days. Then the patient is rested for 7 days before receiving the next cycle which follows the above-described procedures. The cycles are repeated until either the patient has a complete response or when the disease progresses in the patient. A complete response means that the tumor is no longer present. Progression of the cancer means that the tumor starts enlarging or the tumor is no longer stable.

### F. The Objectives of the Trial are:

1. Primary Objectives:
   To evaluate the safety, dose-limiting toxicities (DLTs), and maximum tolerated dose (MTD) of capecitabine administered to patients with advanced, refractory gastrointestinal tract (GI) tumors using a 7 days on +7 days off capecitabine regimen ˜24 hours after a single, fixed, oral 40 mg dose of eniluracil.
   To characterize the pharmacokinetic (PK) profiles of capecitabine, 5 fluorouracil (5 FU), and the quantifiable main metabolites α-fluoro-β-alanine (FBAL), 5′-deoxy-5-fluorocytidine (5′ DFCR), and doxifluridine (5′ DFUR) in plasma.
2. Secondary Objectives:
   To characterize the PK profile of eniluracil and its major metabolite 5-acetyluracil.
   To evaluate dihydropyrimidine dehydrogenase (DPD) enzyme activity over time after a single dose of eniluracil using the ratio of dihydrouracil to uracil as a surrogate to enzyme activity or an analytical method for DPD activity still to be developed.
   To evaluate the effect of eniluracil on QT corrected for heart rate (QTc).
   To evaluate the incidence of adverse events (AE) of special interest (AESI), including the incidence of hand foot syndrome (HFS).
3. Exploratory Objective:
   To gain preliminary evidence of the antitumor activity of eniluracil with capecitabine in advanced GI tumors.

### G. The Endpoints of the Trial are:

1. Primary Endpoints:
   DLTs and incidences of AEs according to NCI CTCAE Version 5.0.
   Concentrations of capecitabine, 5 FU, FBAL, 5′ DFCR, and 5′ DFUR in plasma, PK parameters will be estimated from concentration time data using noncompartmental methods, as data permit.
   Concentrations of eniluracil in plasma; PK parameters will be estimated from concentration-time data using noncompartmental methods, as data permit.
2. Secondary Endpoints:
   To evaluate the incidence of AESI, including the incidence of HFS.
   To evaluate dihydropyrimidine dehydrogenase (DPD) enzyme activity by using the ratio of dihydrouracil to uracil as a surrogate to enzyme activity or a DPD activity analytical method still to be developed.
   The QTc effect of eniluracil treatment will be assessed by comparison of the baseline ECG prior to the first eniluracil dose at Day 1, at 2 hours±30 minutes after the first dose of eniluracil (at the maximum observed concentration [Cₘₐₓ] level), and just before the capecitabine dose on Day 2.
3. Exploratory Endpoints:

Disease assessment according to the standard of care by the investigator every 8 weeks according to RECIST criteria (Version 1.1).
4. Pharmacokinetics Endpoints:

The following PK parameters will be determined for eniluracil, 5-acetyluracil, capecitabine, 5 FU, and the main metabolites FBAL, 5′-DFCR, and 5′-DFUR, data permitting:
Cₘₐₓ: maximum observed plasma concentration;
tmax: the time to reach the peak plasma concentration;
AUC_{0-∞}: area under the plasma concentration-time curve from time zero to infinity;
AUC₀₋ₜ: area under the plasma concentration-time curve from time zero to the last sample with a quantifiable concentration;
AUC0-9: area under the plasma concentration-time curve from time zero to 9 hours;
CL/F: apparent body clearance (calculated for eniluracil and capecitabine only);
Vd/F: apparent volume of distribution (calculated for eniluracil and capecitabine only);
MRT: mean residence time;
λz: terminal elimination rate constant;
t½: terminal elimination half-life; and
other PK parameters will be determined if deemed necessary and if data permit.

### Example 3. Pharmacokinetic (PK) Results

### A. Introduction:

A clinical trial was performed as is generally described above (see, e.g., Example 2) to assess various pharmacokinetic parameters and the safety of capecitabine (discussed below in Example 4) in an effort to corroborate the effects observed in the PDX mouse model (see, e.g., Example 1). Briefly, capecitabine was administered to patients with advanced, refractory gastrointestinal tract (GI) tumors using a 7 days on +7 days off capecitabine regimen ˜24 hours after a single, fixed, oral 40 mg dose of eniluracil. Results obtained in the clinical trial were compared against the data reported in the new drug application for Xeloda used by Roche to obtain FDA market approval (sometimes referred to herein as “the Xeloda study”), which are summarized above in Table 1.

In the Xeloda study, 1255 mg/m² capecitabine was administered twice daily (b.i.d.) and various pharmacokinetic (PK) parameters were assessed in plasma at steady-state (day 14) following administration of the recommended dose of capecitabine (1255 mg/m² b.i.d.) in 8 cancer patients. Specifically, Cₘₐₓ (μg/mL), tₘₐₓ (h), AUC₀₋ₜ (μg*h/mL), AUC_{0-∞}(μg*h/mL), and t_{1/2} (h) were estimated for capecitabine, 5′-DFCR, 5′-DFUR, 5-FU, FUH₂, and FBAL in the Xeloda study.

The results from the Xeloda study are similar to those described in the EMA 2005 Scientific Discussion for Registration of Xeloda in Europe. Briefly, peak plasma concentrations (Cₘₐₓ in μg/mL) for capecitabine, 5′-DFCR, 5′-DFUR, 5-FU, and FBAL were 4.67, 3.05, 12.1, 0.95, and 5.46, respectively, in the 2005 EMA for Xeloda. The time to peak plasma concentrations (tₘₐₓ in hours) were 1.50, 2.00, 2.00, 2.00, and 3.34, respectively, in the 2005 EMA for Xeloda. The AUC_{0-∞}values in μg*h/mL were 7.75, 7.24, 24.6, 2.03, and 36.3, respectively, in the 2005 EMA for Xeloda.

### B. 5-FU:

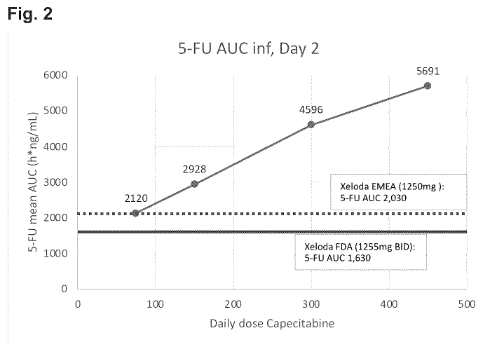

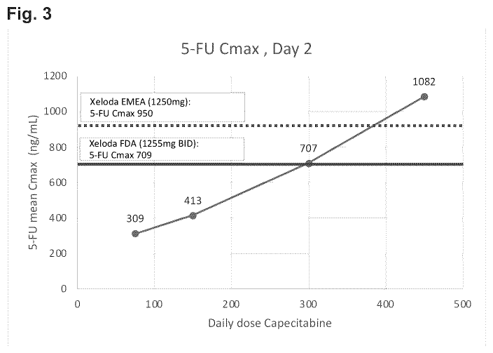

It was determined that 5-FU levels were greatly increased in patients that received a pre-treatment with eniluracil at day 1 followed by treatment with increasing doses of capecitabine at day 2. Both AUC and Cₘₐₓ levels of 5-FU (as shown in FIGS. 2 and 3, respectively) were higher in those who received a pre-treatment with eniluracil compared to levels observed in those receiving capecitabine alone.

Assuming an average body surface of 1.6 m², when capecitabine is given at a dosage of 1255 mg/m², the daily dose for such an average individual is 4016 mg. A single dose of 1250 mg/m² is equivalent to 2000 mg per patient. The AUC_{0-∞} levels observed with capecitabine alone were 1630 h*ng/ml for the 1255 mg/m² b.i.d. dosing and 2030 h*ng/ml for the 1250 mg/m² dosing. The mean AUC_{0-∞} levels observed for the combination of eniluracil and capecitabine at 450 mg daily dose (225 mg b.i.d.) was 5691 h*ng/ml. Therefore, a single dose of 225 mg of capecitabine (in combination with pretreatment with eniluracil) provided between 2.8- to 3.5-fold higher AUC at a total dose that is 8.9-fold lower. Based on the AUC comparison, eniluracil pretreatment amplified the efficacy of capecitabine by a factor of 25- to 30-fold.

Eniluracil potentiation was also observed when analyzing the Cₘₐₓ data, although not as strong as with the AUC data. A dose of 225 mg of capecitabine (in combination with eniluracil pretreatment) yielded a Cₘₐₓ of 1082 ng/mL compared to 709 and 950 ng/ml for capecitabine alone.

Another major difference observed in the PK of 5-FU at day 2 when eniluracil was given in combination with capecitabine was the extension of the half-life (T_{1/2}). The half-life increased from 0.76 hours (capecitabine alone) up to 5.71 hours (capecitabine in combination with eniluracil pretreatment). Extension of the half-life of 5-FU contributed to the increase in the AUC observed for the combination treatment.

TABLE 4

| Capecitabine daily | 5-FU Day 2 | 5-FU Day 2 |
|---|---|---|
| dose (with Eniluracil) | Mean Tₘₐₓ (h) | Mean T_{1/2} (h) |
| 75 | 2 | 3.6 |
| 150 | 2.16 | 3.35 |
| 300 | 3.5 | 3.54 |
| 450 | 3 | 5.71 |
| Capecitabine alone | 2 | 0.76 |

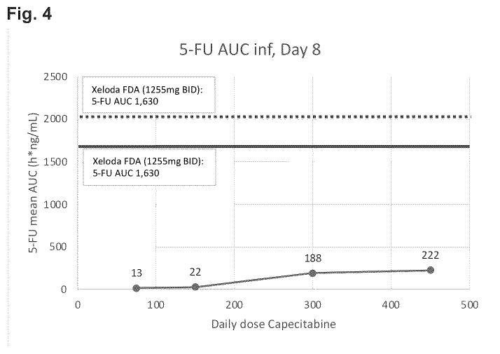

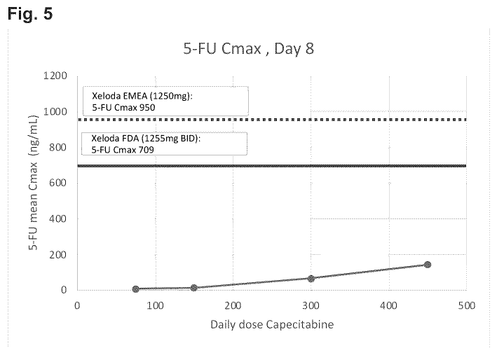

However, at day 8, the effects of eniluracil on the AUC and Cₘₐₓ of 5-FU were no longer observed (as shown in FIGS. 4 and 5, respectively). AUC and Cₘₐₓ levels of 5-FU were reduced, and the half-life and Tₘₐₓ values were comparable to capecitabine treatment alone.

### C. FBAL:

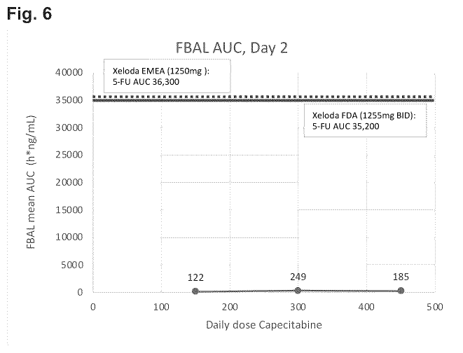

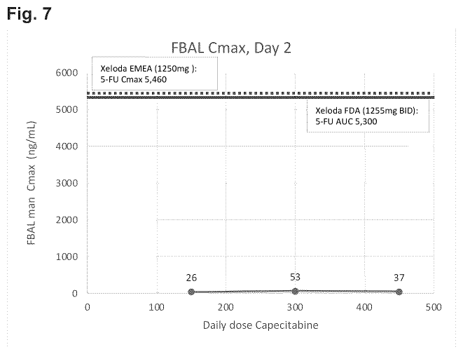

The effect of the pre-treatment with eniluracil on the metabolism of capecitabine into FBAL were, generally, the opposite of those observed for 5-FU. Essentially, while 5-FU exposure was greatly increased by eniluracil, FBAL levels were almost undetectable at day 2. Both AUC and Cₘₐₓ parameters showed that eniluracil completely blocked the metabolism of capecitabine into FBAL, even for the highest doses assessed (as shown in FIGS. 6 and 7, respectively).

Pre-treatment with eniluracil also resulted in an increase in Tₘₐₓ of FBAL at day 2 (7-8 hours compared to 3.24 hours for capecitabine treatment alone).

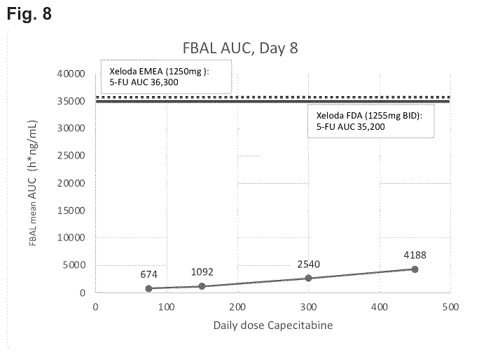

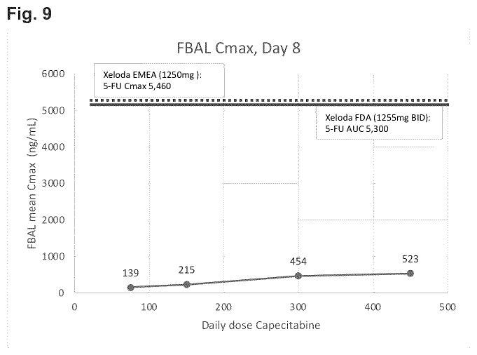

Similar to the results obtained for 5-FU, the effects of eniluracil on FBAL metabolism was no longer apparent at day 8. At day 8, the AUC and Cₘₐₓ and parameters for FBAL were proportional to the levels observed with capecitabine therapy alone (as shown in FIGS. 8 and 9, respectively).

D. Capecitabine and other Metabolites:

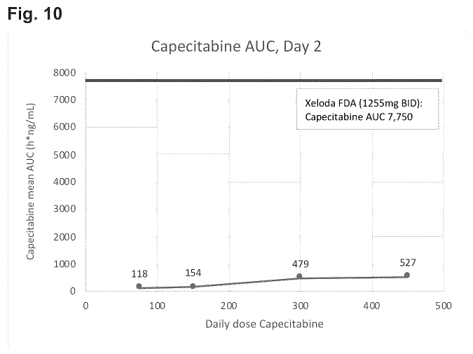

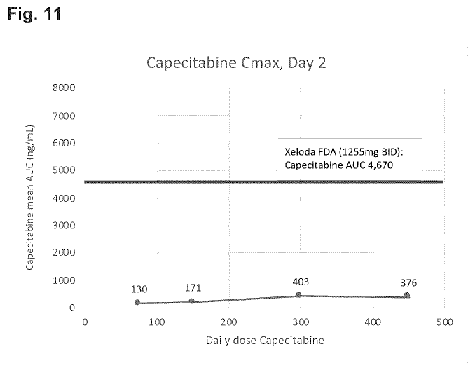

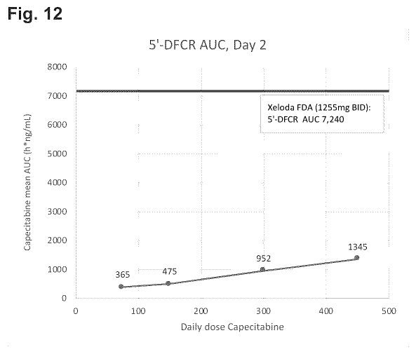

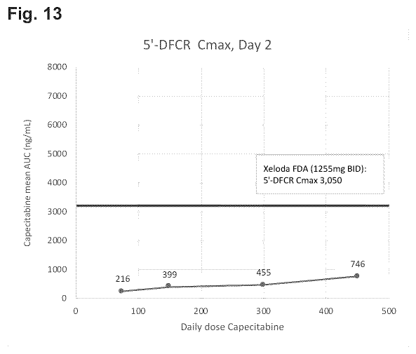

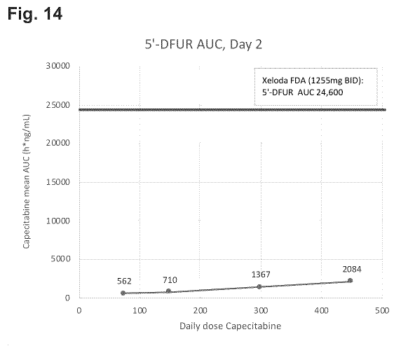

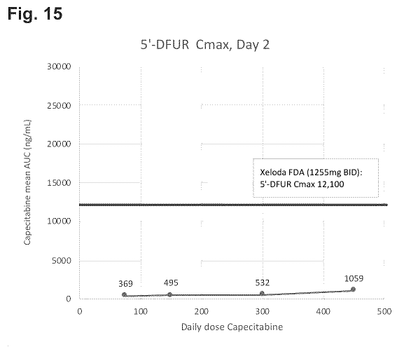

Pretreatment with eniluracil did not significantly affect the PK values of capecitabine, 5—DCFR, and 5′DFUR, since the AUC and Cₘₐₓ values (Capecitabine—FIGS. 10 and 11, respectively; DFCR—FIGS. 12 and 13, respectively; DFUR—FIGS. 14 and 15, respectively) when capecitabine was administered at a daily dose of 450 mg are approximately 10- to 15-fold lower when compared to capecitabine treatment (alone) at an estimated daily dose of about 4000-4500 mg (1255 mg/m² b.i.d.).

### E. Variability of PK Parameters:

As mentioned in the FDA and EMA regulatory submissions for Xeloda, the variability of PK parameters (e.g., AUC and Cₘₐₓ) for capecitabine and its metabolites was very high. This high variability occurred in patients in which capecitabine had been dose-adjusted based on their body surface (body surface adjustment (BSA)). One problem associated with capecitabine in clinical practice is the need to individualize the treatment doses for each patient.

In the current clinical trial, capecitabine was given to patients as a fixed dose, and as such was not individually adjusted. The data generated for 5-FU at day 2 shows a remarkably low level of variability (15-30%).

TABLE 5

| Capecitabine daily | 5-FU mean Cₘₐₓ | |
|---|---|---|
| dose | Day 2 (ng/ml) | Standard Deviation |
| 75 mg | 309 | NA |
| 150 mg | 413 | 78 |
| 300 mg | 707 | 160 |
| 450 mg | 1082 | 293 |

TABLE 6

| Capecitabine daily | 5-FU mean AUCₗₐₛₜ | |
|---|---|---|
| dose | Day 2 (h*ng/ml) | Standard Deviation |
| 75 mg | 1600 | NA |
| 150 mg | 2252 | 431 |
| 300 mg | 3880 | 953 |
| 450 mg | 6583 | 2299 |

### Example 4. Safety and Efficacy Results

### A. Introduction:

A clinical trial was performed as is generally described above (see, e.g., Example 2) to assess various pharmacokinetic parameters (discussed above in Example 3) and the safety of capecitabine in an effort to corroborate the effects observed in the PDX mouse model (see, e.g., Example 1). Briefly, capecitabine was administered to patients with advanced, refractory gastrointestinal tract (GI) tumors using a 7 days on +7 days off capecitabine regimen ˜24 hours after a single, fixed, oral 40 mg dose of eniluracil.

### B. Patient Demographics, Cancer Type, and Number of Cycles:

A summary of the patient cohorts (1, 2A, 3, and 4) is shown below in Table 7. All patients had late-stage cancers and, with the exception of one patient in cohort 3, completed more than 2 cycles of treatment at the cutoff used for data collection.

TABLE 7

| | | | | | Dose | | # of |
|---|---|---|---|---|---|---|---|
| | Patient | | Age | | of | | Cycles |
| Cohort | # | Gender | (yrs) | Cancer type | PCS6422 | Dose of Capecitabine | completed |
| 1 | 102-101 | Male | 59 | Stage IIIB | 40 mg Day 1 | 75 mg QD, Days 2-8 for | 24 |
| | | | | colorectal cancer | | first 7 cycles, then 75 | |
| | | | | | | mg BID Day 2-8 for | |
| | | | | | | remaining cycles | |
| 2A | 103-102 | Female | 69 | Stage IV pancreatic | 40 mg Day 1 | 75 mg BID, Days 2-8 | 4 |
| | | | | adenocarcinoma | | | |
| 2A | 104-103 | Male | 68 | Stage IV gastric | 40 mg Day 1 | 75 mg BID, Days 2-8 | 8 |
| | | | | adenocarcinoma | | | |
| 2A | 105-104 | Female | 50 | Stage IV colorectal | 40 mg Day 1 | 75 mg BID, Days 2-8 | 4 |
| | | | | cancer | | | |
| 3 | 105-113 | Female | 66 | Stage IV | 40 mg Day 1 | 150 mg BID, Days 2-8 | 4 |
| | | | | appendiceal | | | |
| | | | | adenocarcinoma | | | |
| 3 | 107-114 | Male | 49 | Stage IIIb | 40 mg Day 1 | 150 mg BID, Days 2-8 | 1 |
| | | | | colorectal cancer | | | |
| 3 | 107-115 | Female | 66 | Stage IV colorectal | 40 mg Day 1 | 150 mg BID, Days 2-8 | 7 |
| | | | | cancer | | | |
| 3 | 107-116 | Male | 65 | Colorectal cancer | 40 mg Day 1 | 150 mg BID, Days 2-8 | 5 |
| 4 | 107-117 | Male | 62 | Stage IIIb rectal | 40 mg Day 1 | 225 mg BID, Days 2-8 | 6 |
| | | | | cancer | | | (ongoing) |
| 4 | 103-118 | Male | 43 | Stage IV sigmoid | 40 mg Day 1 | 225 mg BID, Days 2-8 | 2 |
| | | | | color cancer | | | |
| 4 | 105-119 | Female | 70 | Stage IV pancreatic | 40 mg Day 1 | 225 mg BID, Days 2-8 | 3 |
| | | | | adenocarcinoma | | | |

C. Adverse Events with Xeloda/Capecitabine:

The Xeloda label contains the following warnings and precautions:
“Acute renal failure secondary to dehydration can be fatal. If Grade 2 (or higher) dehydration occurs, XELODA treatment should be immediately interrupted and the dehydration corrected . . . .”
“Similar to that of other fluorinated pyrimidines sudden death due to cardiotoxicity has been observed with XELODA . . . .”
“XELODA can induce severe skin reactions such as hand-and-foot syndrome, Stevens-Johnson syndrome and Toxic Epidermal Necrolysis. If grade 2 (or higher) event occurs, administration of XELODA should be immediately interrupted . . . .”
“Rarely, unexpected, severe toxicity (e.g. stomatitis, diarrhea, mucosal inflammation, neutropenia and neurotoxicity) associated with 5-FU has been attributed to a deficiency of DPD activity. Fatalities have been reported . . . .”
“Altered coagulation parameters and/or bleeding have been reported in patients taking XELODA concomitantly with coumarin-derived anticoagulants such as warfarin. Patients taking coumarin-derivative anticoagulants concomitantly with XELODA should be monitored regularly for alterations in their coagulation parameters (PT or INR) and the anticoagulant dose adjusted accordingly . . . .”

Furthermore, it contains the following statement: “If toxicity on therapy occurs, XELODA should be interrupted until the event resolves, or the severity decreases when the following toxicities occur at a severity of grade 2 or greater: diarrhea, hand-foot syndrome, nausea, hyperbilirubinemia, vomiting or stomatitis . . . .”

Table 8 below details the most frequently observed adverse drug reactions in the clinical trial for registration of Xeloda.

TABLE 8
Summary of ADRs Reported in ≥5% of Patients with Colon Cancer Tested
with XELODA Monotherapy or i.v. 5-FU/LV in the Adjuvant Setting.

| | Adverse Event | | | |
|---|---|---|---|---|
| | XELODA 1250 mg/m²/bid | | i.v. 5-FU/LV* | |
| | (n = 995) | | (n = 974) | |
| | Total | Grade 3/4 | Total | Grade 3/4 |
| Body System/Adverse Event | % | % | % | % |
| Gastrointestinal | | | | |
| Diarrhea | 46 | 11 | 64 | 13 |
| Stomatitis | 22 | 2 | 60 | 14 |
| Nausea | 33 | 2 | 47 | 2 |
| Vomiting | 14 | 2 | 20 | 1 |
| Abdominal pain | 10 | 2 | 13 | 1 |
| Constipation | 6 | — | 7 | <1 |
| Abdominal pain upper | 6 | <1 | 5 | <1 |
| Dyspepsia | 5 | <1 | 4 | — |
| Skin and Subcutaneous | | | | |
| Hand-foot Syndrome** | 60 | 17 | 9 | <1 |
| Alopecia | 6 | — | 22 | <1 |
| Rash | 6 | — | 8 | — |
| Erythema | 6 | 1 | 5 | <1 |
| General Disorders | | | | |
| Fatigue | 15 | <1 | 15 | 1 |
| Lethargy | 10 | <1 | 9 | <1 |
| Asthenia | 9 | <1 | 9 | 1 |
| Pyrexia | 4 | <1 | 6 | <1 |
| Nervous System Disorders | | | | |
| Dysgeusia | 6 | — | 9 | — |
| Dizziness | 5 | <1 | 4 | — |
| Metabolism and Nutrition | | | | |
| Disorders | | | | |
| Anorexia | 9 | <1 | 10 | <1 |
| Eye | | | | |
| Conjunctivitis | 5 | <1 | 5 | <1 |
| Blood and Lymphatic System | | | | |
| Neutropenia | 2 | <1 | 8 | 5 |

*Mayo Clinic regimen
**Based on the post-marketing experience, persistent or severe palmar-plantar erythrodysaesthesia syndrome (grade 2 and above) can eventually lead to loss of fingerprints (see WARNINGS AND PRECAUTIONS).

### D. Adverse Events in Clinical Trial:

Table 9 below shows adverse events (grade 2 or higher) observed in cohorts 1, 2A, 3, and 4.

TABLE 9

| | 75 mg day | | 150 mg day | | 300 mg day | | 450 mg day | |
|---|---|---|---|---|---|---|---|---|
| | Cohort 1 | | Cohort 2A | | Cohort 3 | | Cohort 4 | |
| Adverse | Grade | Grade | Grade | Grade | Grade | Grade | Grade | Grade |
| Event | 2 | 3/4 | 2 | 3/4 | 2 | 3/4 | 2 | 3/4 |
| Diarrhea | 0 | 0 | 0 | 0 | 0 | 0 | 0 | 0 |
| Stomatitis | 0 | 0 | 0 | 0 | 0 | 0 | 0 | 0 |
| Nausea | 0 | 0 | 0 | 0 | 0 | 0 | 0 | 0 |
| Vomiting | 0 | 0 | 0 | 0 | 0 | 0 | 0 | 0 |
| Other GI | 0 | 0 | 0 | 0 | 0 | 0 | 0 | 0 |
| Hand-foot | 0 | 0 | 0 | 0 | 0 | 0 | 0 | 0 |
| Syndrome | | | | | | | | |
| Other Skin | 0 | 0 | 0 | 0 | 0 | 0 | 0 | 0 |
| Fatigue | 0 | 0 | 0 | 0 | 0 | 1/3 | 0 | 0 |
| Other | 0 | 0 | 1/3 | 0 | 0 | 0 | 0 | 0 |
| General | | | | | | | | |
| Nervous | 0 | 0 | 0 | 0 | 2/3 | 1/3 | 0 | 0 |
| System | | | | | | | | |
| Neutropenia | 1/1 | 0 | 0 | 0 | 1/3 | 0 | 0 | 1/3 |
| Other | 0 | 0 | 1/3 | 1/3 | 1/3 | 0 | 0 | 0 |
| Hematologic | | | | | | | | |

Although the number of patients treated for each dose was limited, making any safety analysis difficult, it was notable that no grade 2 HFS, diarrhea, nausea, vomiting, or stomatitis were observed in the 11 patients treated with the combination of eniluracil and capecitabine at the various doses assessed. These grade 2 AEs are indicated in the Xeloda label and are the main reason for treatment delay, interruption, and dose reduction for Xeloda (capecitabine). Since HFS was observed in about 60% of the patients and diarrhea in about 46% of the patients in the Xeloda clinical trials, the total absence of grade 2 AEs in the current clinical trial was very encouraging in view of the fact that many patients were treated for multiple cycles of therapy.

### E. Preliminary Response Rate:

Table 10 below contains some data regarding the RECIST analysis of the observed lesion(s) for patients enrolled in the clinical trial.

TABLE 10

| | | Cycles | |
|---|---|---|---|
| Patient | Dose | completed | Overall Response |
| 102-101 | 75 mg Day/BID | 24 | Stable Disease (SD) |
| 103-102 | 75 mg BID | 4 | Progressive Disease (PD) |
| 104-103 | 75 mg BID | 8 | SD for 5 cycles, PD at cycle 9 |
| 105-104 | 75 mg BID | 4 | PD |
| 105-113 | 150 mg BID | 4 | PD |
| 107-114 | 150 mg BID | 1 | Not available |
| 107-115 | 150 mg BID | 7 | SD for 8 cycles, ongoing |
| 107-116 | 150 mg BID | 5 | SD for 5 cycles, ongoing |
| 107-117 | 300 mg BID | 6 | Not available |
| 103-118 | 300 mg BID | 2 | Not available |
| 105-119 | 300 mg BID | 3 | Not available |

As the clinical trial is still ongoing, it was too early to make any meaningful consideration on clinical efficacy. The patient in cohort 1 (102-101), dosed with 75 mg capecitabine daily for the first 7 cycles and then 75 mg b.i.d. for the remaining cycles, has been treated for a total of 24 cycles, corresponding to almost 1 year of therapy. During this time, the patient exhibited stable disease. Further, patients 107-115 and 107-116 were treated for multiple cycles and showed stable disease.

### REFERENCES

[1] Schilsky et al., 1998, J. Clin Oncol 4:1450-7.
[2] Schüller J, et al., 2000, Cancer Chemother Pharmacol. 291-7.
[3] FDA, U.S. 2001. Drug Approval Package: Xeloda. 4 30. www.accessdata.fda.gov/drugsatfda_docs/nda/2001/20896s6_Xeloda.cfm
[4] Roche Canada. 2017. “Xeloda (capecitabine) Product Monograph.” 1-59.
[5] Rivera E, et al. 2014, Clin. Breast Cancer 14 (1): 26-30.
[6] Fiebig HH: Comparison of Tumor Response in Nude Mice and in Patients. In: Winograd B, Peckham MJ, Pinedo HM (eds.), Human Tumour Xenografts in Anticancer Drug Development. Berlin, Springer, 1988, 25-30.
[7] Fiebig HH, et al: Combined in Vitro/in Vivo Test Procedure with Human Tumor Xenografts for New Drug Development. In: Fiebig HH, Berger DP (eds.), Immunodeficient Mice in Oncology. Contrib. Oncol. Basel, Karger, 1992, 42: 321-351.
[8] Schueler J, et al: Patient-derived renal cell carcinoma xenografts exhibit distinct sensitivity patterns in response to antiangiogenic therapy and constitute a suitable tool for biomarker development. Oncotarget, 2018, 9(57): 30946-30961.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed is:

1. A method for treating cancer in a human subject, comprising:
(a) orally administering eniluracil at a dose 30-60 mg/day to the subject;
(b) at least after 10 hours of step (a), orally administering a fixed dose of capecitabine at about 140-700 mg/day to the subject for 2 to 14 consecutive days or a dose of capecitabine providing a 5-FU exposure (AUC) of approximately 1500 h*ng/ml or higher; and
(c) 3 to 14 days after step (b), repeat the steps (a) and (b).

2. The method of claim 1, wherein eniluracil is administered at about 40 mg/day.

3. The method of claim 1, wherein capecitabine is administered at about 160-500 mg/day.

4. The method of claim 1, wherein step (b) occurs at least after 17 hours of step (a).

5. The method of claim 1, wherein step (b) occurs 10-24 hours after step (a).

6. The method of claim 1, wherein step (c) occurs 7 days after step (b).

7. The method of claim 1, where the cancer is colorectal cancer, gastrointestinal tract tumor, breast cancer, pancreatic cancer, head and neck cancer, lung cancer, or advanced biliary tract cancer.
